SEQUENCE LISTING (12 sequences)

SEQ ID NO: 1 - protein, 245 aa, Bacillus cereus
WSAEDKHKEGVNSHLWIVNRAIDIMSRNTTLVKQDRVALLNEWRTELENGIYAADYENPYYDNSTFASHFYDPDNGKTYIPYAKQAKETGAKYFKLAGESYKNKDMKQAFFYLGLSLHYLGDVNQPMHAANFTNLSYPQGFHSKYENFVDTIKDNYKVTDGNGYWNWKGTNPEDWIHGAAVVAKQDYAGIVNDNTKDWFVRAAVSQEYADKWRAEVTPMTGKRLMDAQRVTAGYIQLWFDTYGDR

SEQ ID NO: 2 - protein, 245 aa, synthetic construct
WSAEDPHNEGVNTHLWIVNRAIDIMARNTTVVKQNAVALLNEWRTELEQGIYDADYENPYYDNSTFASHFYDPDTGKTYIPEAKNAKTTGAKYFKLAGEAYQNKDFEKAFYYLGLSLHYFTDVNQPMHAANFTNISYPQGFHSKYENYVDTIKDNYKVTDGNGYWNWKSDNPEEWIHEAAVAAKADYPKIVNDKTKKWFLKAAVSQDYADKWRAEVTPATGKRLMEAQRITAGYIHLWFDTYVNN

SEQ ID NO: 3 - protein, 245 aa, synthetic construct
WSAEDKHKEGVNSHLWIVNRAIDIMSRNTTLVKQDRVALLNEWRTELENGIYAADYENPYYDNSTFASHFYDPDNGKTYIPYAKNAKTTGAKYFKLAGESYKNKDFKQAFFYLGLSLHYFTDVNQPMHAANFTNLSYPQGFHSKYENFVDTIKDNYKVTDGNGYWNWKGTNPEDWIHGAAVVAKQDYPGIVNDNTKDWFVRAAVSQEYADKWRAEVTPMTGKRLMDAQRITAGYIQLWFDTYGDR

SEQ ID NO: 4 - protein, 245 aa, synthetic construct
WSAEDKHKEGVNSHLWIVNRAIDIMSRNTTLVKQDRVAQLNEWRTELENGIYAADYENPYYDNSTFASHFYDPDNGKTYIPFAKNAKTTGAKYFKLAGESYKNKDMKQAFFYLGLSLHYLGDVNQPMHAANFTNLSYPQGFHSKYENFVDTIKDNYKVTDGNGYWNWKGTNPEEWIHGAAVVAKQDYSGIVNDNTKDWFVKAAVSQEYADKWRAEVTPMTGKRLMDAQRITAGYIQLWFDTYGDR

SEQ ID NO: 5 - protein, 245 aa, synthetic construct
WSAEDKHKEGVNSHLWIVNRAIDIMSRNTTLVKQDRVALLNEWRTELENGIYAADYENPYYDNSTFASHFYDPDNGKTYIPYAKNAKTTGAKYFKLAGESYKNKDFKQAFFYLGLSLHYFGDVNQPMHAANFTNLSYPQGFHSKYENFVDTIKDNYKVTDGNGYWNWKGTNPEDWIHGAAVVAKQDYPKIVNDNTKDWFVRAAVSQEYADKWRAEVTPMTGKRLMDAQRVTAGYIQLWFDTYGDR

SEQ ID NO: 6 - protein, 245 aa, synthetic construct
WSAEDKHKEGVNSHLWIVNRAIDIMSRNTTLVKQDRVALLNEWRTELENGIYAADYENPYYDNSTFASHFYDPDNGKTYIPYAKNAKTTGAKYFKLAGESYKNKDFKQAFFYLGLSLHYFTDVNQPMHAANFTNLSYPQGFHSKYENFVDTIKDNYKVTDGNGYWNWKGTNPEDWIHGAAVVAKQDYPKIVNDNTKDWFVRAAVSQEYADKWRAEVTPMTGKRLMDAQRVTAGYIQLWFDTYGDR

SEQ ID NO: 7 - protein, 245 aa, Bacillus thuringiensis
WSAEDKHKEGVNSHLWIVNRAIDIMSRNTTLVKQDRVAQLNEWRTELENGIYAADYENPYYDNSTFASHFYDPDNGKTYIPFAKQAKETGAKYFKLAGESYKNKDMKQAFFYLGLSLHYLGDVNQPMHAANFTNLSYPQGFHSKYENFVDTIKDNYKVTDGNGYWNWKGTNPEDWIHGAAVVAKQDYSGIVNDNTKDWFVKAAVSQEYADKWRAEVTPMTGKRLMDAQRVTAGYIQLWFDTYGDR

SEQ ID NO: 8 - protein, 245 aa, Bacillus thuringiensis
WSAEDKHNEGINSHLWIVNRAIDIMSRNTTIVNPNETALLNEWRADLENGIYSADYENPYYDDSTYASHFYDPDTGTTYIPFAKHAKETGAKYFNLAGQAYQNQDMQQAFFYLGLSLHYLGDVNQPMHAASFTDLSYPMGFHSKYENFVDTIKNNYIVSDSNGYWNWKGANPEDWIEGAAVAAKQDYPGVVNDTTKDWFVKAAVSQEYADKWRAEVTPVTGKRLMEAQRVTAGYIHLWFDTYVNR

SEQ ID NO: 9 - protein, 319 aa, Streptomyces antibioticus
MEPAATTYGTSTSVGVHNAYEKEKYRYFADALDSGAALLELDLWSNALGRSWRVSHSNPLGNNSNCEGAANASELRTKSRDQDFAGCLSDMRAWHDAHPGHRPILLKIEMKDGFNAKGGRGPAEFDALIRQKLGDAVYGPGDLTGGHATADEAVRAGGWPSRADLAGKFLFELIPGTVEEKNPFDKLWTDVEYAGHLKDLAAQGKLAQSTAFPAVHGAAPGDPRERYADPALRPWFVVFDGDAPTYLNGSIDTSWYDTRHYLLIMTDAHNVPPVIDGTHPTEAEALARVRQLAAAHASFATADWYPLPSVLKTVVPRGA

SEQ ID NO: 10 - DNA, 861 bp, synthetic construct
  source 1..861: mol_type = other DNA
catatgaaaaagaaggtcctggcattggcagcggctattaccctggtcgcaccgttgcagtctgtcgcattcgcacacgagaacgatggcggccagcgcttcggcgtgatcccacgttggtctgcagaggacccacacaacgaaggcgttaacacccacctgtggatcgttaaccgcgctatcgacatcatggcccgcaacaccaccgtcgtgaagcagaacgcggtcgcactgcttaacgaatggcgcaccgaactggagcagggtatctacgacgcagattacgaaaacccatactacgacaactccaccttcgcctcacacttctacgacccagacactggcaagacctacattccggaggcaaagaacgcaaagactaccggcgcgaagtacttcaagcttgctggtgaagcttaccagaacaaggacttcgagaaggcattctactacctgggtctcagcctgcactacttcaccgatgtgaaccagccaatgcacgcagctaacttcaccaacattagctacccacagggattccactccaagtatgagaactatgtcgataccatcaaggacaactacaaggtgaccgatggtaacggctactggaactggaagtccgacaacccagaggagtggatccacgaagcagctgttgcagctaaggctgactacccaaagattgtgaacgacaagaccaagaagtggttcctgaaggcggcagtttcccaggactacgcagacaagtggcgcgctgaggttacgccagcaaccggcaagcgccttatggaagcacagcgcatcaccgctggctacatccacctctggttcgatacctacgttaacaactaagaattc

SEQ ID NO: 11 - protein, 245 aa, synthetic construct
WSAEDPHNEGVNTHLWIVNRAIDIMARNTTVVKQNAVALLNEWRTELEQGIYDADYENPYYDDSTFASHFYDPDTGKTYIPEAKNAKTTGAKYFKLAGEAYQNKDFEKAFYYLGLSLHYFTDVNQPMHAASFTDISYPQGFHSKYENYVDTIKDNYKVTDGNGYWNWKSDNPEEWIHEAAVAAKADYPKIVNDKTKKWFLKAAVSQDYADKWRAEVTPATGKRLMEAQRITAGYIHLWFDTYVNN

SEQ ID NO: 12 - DNA, 738 bp, synthetic construct
  source 1..738: mol_type = other DNA
tggtctgctgaagacccccataatgagggtgtgaacacccacttatggatcgttaaccgtgccattgacataatggctcgtaatactactgtcgttaagcaaaatgctgtcgctttgctaaacgaatggcgtactgaattggagcaaggaatctatgatgctgattacgaaaacccatactatgatgattccaccttcgcttcacatttttacgacccagacacaggtaagacatatatacctgaggctaagaatgctaagactactggtgctaaatacttcaagttggctggagaggcttatcagaacaaggattttgaaaaagctttttattacttaggtttgtctttgcactacttcactgacgtaaaccagccaatgcacgctgcatctttcactgacatctcctatcctcaaggattccattcaaaatatgagaattatgttgatacgattaaagataattacaaggttacagatggaaacggatactggaactggaagagtgacaacccagaagaatggattcacgaagccgctgttgctgctaaggccgactatccaaaaattgtgaacgacaaaactaaaaagtggtttcttaaagcagcagttagtcaagattatgcagataaatggcgagctgaggtcacaccagctaccggaaagagattaatggaagcccaaagaattacagctggttatatacacctatggttcgatacgtacgttaacaactaa

(19) United States
(12) Patent Application Publication
(10) Pub. No.: US 2025/0346872 A1
(43) Pub. Date: Nov. 13, 2025

(54) MUTATED PHOSPHOLIPASE C ENZYME

(71) Applicant: KECLON SA, Rosario - Santa FE (AR)

(72) Inventors: Salvador PEIRU, Rosario - Santa FE (AR); Hugo MENZELLA, Rosario - Santa FE (AR); Luciano Andrés ABRIATA, Renens, Vaud (CH); Maria Eugenia CASTELLI, Rosario - Santa FE (AR)

(21) Appl. No.: 18/727,871

(22) PCT Filed: Jan. 12, 2023
(86) PCT No.: PCT/IB2023/050278

§ 371 Date: Jul. 10, 2024

Related U.S. Application Data
(60) Provisional application No. 63/299,510, filed on Jan. 14, 2022.

(51) Int. Cl. C12N 9/16; A23D 9/04; C11B 3/00

(52) U.S. Cl. CPC C12N 9/16; A23D 9/04; C11B 3/003; C12Y 301/04003

(57) ABSTRACT

A mutated phospholipase C enzyme, comprising an amino acid sequence wherein at least one amino acids is substituted in the position selected from the group consisting of 120, 85, 88, 106, 121, 188, 189, 230, 53, 82, 178 and 194 of the amino acid sequence of SEQ ID No. 1, or an amino acid sequence with at least 80% identical of SEQ ID No. 1.

### CROSS REFERENCE TO RELATED APPLICATIONS

This application is a 371 of PCT/IB2023/050278 filed on Jan. 12, 2023, which claims the benefit of U.S. Provisional Patent Application No. 63/299,510 filed on Jan. 14, 2022, the contents of each application are incorporated herein by reference.

### DESCRIPTION

### Background

Vegetable oils represent a major sector of the global economy and the demand for food, and fuel production is continuously increasing. Even when they represent a source for renewable fuels, the environmental impact of deforestation and the waste generated makes the production unsustainable.

Current soybean refining processes generates up to 70 kg of waste per tonne of crude oil, which is inadequately disposed and cause environmental pollution, particularly in countries with poor regulations and controls (Bailis R, et., al., Biofuels 5 (5): 469-485; 2014 and Selfa et al. 2015). Thus, there is an urgent need for environmentally friendly oil refining methods; Alexandre V M F et al, (2016) Minimizing solid wastes in an activated sludge system treating oil refinery wastewater. Chem Eng Process 103:53-62, Gupta M et al, (2017) Practical guide to vegetable oil processing AOCS Press 2nd edition, Wang L K, et al, (2004) Handbook of industrial and hazardous wastes treatment. CRC Press.

Crude vegetable oils possess up to 3.5% of phospholipids (PLs), equivalent to 1200 ppm of inorganic phosphorus (P), that need to be removed in the first refining step, known as “degumming”. This process removes PLs from crude oil, which causes the major losses of oil and generates large amounts of waste, is known as “water degumming” (Dumont M-J, et., al., Food Res Int 40:957-974, 2007). Here, water is added to extract ˜35 kg (per ton of crude oil, dry bases) of a heavy phase (“gums”) composed by PLs and trapped TAGs by centrifugation. The resulting degummed oil contains 100-200 ppm of P and is in many countries exported as is to be further processed to obtain edible oil or biodiesel (Hails, G. et al. Appl Microbiol Biotechnol 104, 7521-7532 (2020).

In the last decade, enzymatic degumming processes using type C phospholipases (PLCs) were implemented. PLC enzymes hydrolyze vegetable oils phospholipids to oil-soluble diacylglycerol and water-soluble phosphate esters. The produced diacylglycerols remain in the oil during refining which contribute to increasing the oil yield. In addition, the amount of gums is reduced and less oil is retained, also contributing to an improved oil yield. The overall extra-yield provided by the treatment can be higher than 2%, depending on the amount and class of PLs present in the crude oil. For soybean oil, PLCs with specificity for both phosphatidylcholine (PC) and phosphatidylethanolamine (PE) are the most effective enzymes, since together represent ˜70% of the PLs present in soybean oil (Cerminati, S., et., al., (2017) Appl Microbiol Biotechnol 101, 4471-4479; Cerminati, S., et., al., Appl Microbiol Biotechnol 102, 6997-7005 (2018): Dijkstra, A. J., Eur. J. Lipid Sci. Technol, 2010. 112: p. 1178-1189; Elena, C., et., al., (2017) Process Biochemistry 54, 67-72; Elena, C., et., al., (2016) Process Biochemistry 51, 1935-1944; Hammond E G, et., al., (2005) Soybean oil Bailey's industrial oil and fat products. Wiley; Sein A, et., al., (2019) Enzymes in vegetable oil degumming processes. In: Vogel A, May O (eds) Industrial enzyme applications, pp 323-350.

Despite its benefits, the use of PLC-based technologies is still scarce. A major barrier for early adopters is the capital investment required to modify the existing crushing plants, to satisfy the strict and narrow working conditions required by the enzymes. Today, PLCs in the market are active at 55° C. but with low efficiency and requiring a long period of time to react with its substrates. Since the crude oil is extracted at 75-85° C., and the separation of gums by centrifugation is more efficient at ˜80° C., the insertion of an enzymatic degumming process requires heat exchangers before and after the 55° C. degumming process, with additional piping and mixers. Fouling of the heat exchangers by the gums present in crude oil, leads to higher operating costs, which contributes to discourage the adoption of the enzymatic degumming by an industrial sector typically reluctant to disrupt new technologies. Also, it is possible to find other variants of PLC, like the one disclosure in the patent application US2022064611, WILMAR SHANGHAI BIOTECHNOLOGY RES & DEV CT CO LTD, which describes a new variant of a PLC enzyme that can be used in a degumming process but does not avoid the implementation of a step of lower the temperature during the process.

Other companies teachs enzymes that can reduce the presence of gums in an oil degumming process, for example the U.S. Pat. No. 10,351,795B2 (Novozymes AS) and a phospholipase C as present in the product Purifine PLC, sold by DSM (see Patent Application WO2016166149A1, incorporated as reference). These enzymes still show the need of maintain a good enzymatic activity at temperature over 60° C. temperature.

So there is a need to provide an enzymatic degumming process without the requirements of heat exchangers before and after the degumming process.

### Enhancing the Thermostability of Enzymes by Consensus Based Engineering Methods

There are different methods for obtaining highly thermostable proteins. There is no dominant mechanism for the thermostability of proteins. Several substitutions with a positive effect have been reported, including “core packing”, electrostatic effect due to increased salt bridges, stiffening of enzymes by substitution of proline residues in loop regions, increased hydrogen bonds, pi-pi stacking of chains lateral and lower number of thermolabile residues. Alternatively, covalent binding sites on the enzyme can be increased for stability by immobilization.

Rational design requires extensive knowledge of the enzyme to be optimized such as its crystal structure, knowledge of the denaturation mechanism, and an idea of the weak points of the enzyme. The number of potential substitutions that can be made in a given protein is very large, which makes it difficult to rationally choose the residues to be modified. However, it is less time consuming than a random strategy as a limited number of variants are created.

Combinatorial protein design, also called directed evolution, is based on the generation of diversity followed by selection or screening to identify the variant with the desired properties (Arnold, F. H. and A. A. Volkov, Directed evolution of biocatalysts. Curr Opin Chem Biol, 1999. 3 (1): p. 54-9.). This method is laborious, but does not require as much detailed information as rational design.

Another method, the semi-rational design combines the advantages of both previous methods to reduce the number of variants to generate and the amount of information required and increase the proportion of successful variants generated.

The consensus mutation method can be considered as a semi-rational method. In nature, protein families have developed as a result of a continuous process of random mutagenesis, tending to eliminate the most destabilizing mutations (Kimura, M., Recent development of the neutral theory viewed from the Wrightian tradition of theoretical population genetics. Proc Natl Acad Sci USA, 1991. 88 (14): p. 5969-73). As a result, the amino acids that stabilize a protein tend to be more prevalent than other amino acids at a given position in a protein family.

It has been shown for several proteins that consensus mutations, where a particular amino acid of a specific protein is replaced by the most common amino acid present at that position within members of that family, frequently result in stabilized variants of the protein (Steipe, B., et al., Sequence statistics reliably predict stabilizing mutations in a protein domain. J Mol Biol, 1994. 240(3): p. 188-9216).

The semi-rational construction of libraries based on multiple sequence alignments of a protein with its natural counterparts has been described. The resulting combinatorial library contains a large fraction of stabilized mutants and eliminates the need for selective enrichment or robotics-based techniques and allows the identification of stabilized variants by evaluating a moderate number of variants on microplates (on the order of 10³). As an alternative to this strategy, combinatorial libraries based on the consensus sequence have been constructed demonstrating that it results in an efficient method for the rapid identification of stabilizing consensus mutations. But it is important to remark that not all of the consensus mutations enhance the thermostability of the protein, and some of the consensus mutations should be avoided.

Amin et. al., showed that combinatorial consensus mutagenesis technique can generate mutants more stable than the parental protein (Amin, N., et al., Protein Eng Des Sel, 2004. 17(11): p. 787-93, incorporated as reference).

In nature, protein families arise as a result of the continuous process of random mutagenesis, recombination and selection, which tends to select most stabilizing amino acid changes. Thus, at any given position in a protein family, residues that stabilize a protein prevail over other amino acids. Consensus mutagenesis, where a particular amino acid of a protein is substituted by the most common amino acid that is present in that position among the sequences in a given protein domain, tend in most cases to stabilize the resulting synthetic proteins (Steipe, B., J Mol Biol, 1994. 240 (3): p. 188-92, 1994).

Unfortunately, PC PLCs with both the required catalytic efficiency and the capability to tolerate temperatures near 80° C. have not been reported so far.

### SUMMARY OF THE INVENTION

An object of the present invention is to provide a mutated phospholipase C enzyme comprising an amino acid sequence wherein at least one amino acid is substituted in a position selected from the group consisting of 120, 85, 88, 106, 121, 188, 189, 230, 53, 82, 178 and 194 of: the amino acid sequence of SEQ ID No. 1 or an amino acid sequence with at least 80%, 85%, 90%, 95%, 97% or 98% identical of SEQ ID No. An embodiment of the present invention wherein said mutated phospholipase C enzyme said at least substitution is selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1. In a more preferred embodiment of the present invention said substitution is selected from the group consisting of L120F, Q85N, E88T, M106F, G121T, A188P, G189K, V230I, A53D, Y82E, G178E and N194K of: the amino acid sequence of SEQ ID No. 1 or an amino acid sequence with at least 80%, 85%, 90%, 95%, 97% or 98% identical of SEQ ID No. 1.

Another embodiment of the present invention, wherein said mutated phospholipase C enzyme comprises the amino acid substitution 120F. Another embodiment of the present invention, wherein said mutated phospholipase C enzyme comprises the amino acid substitution 85N; another embodiment of the present invention, wherein said mutated phospholipase C enzyme comprises the amino acid substitution 88T; another embodiment of the present invention, wherein said mutated phospholipase C enzyme comprises the amino acid substitution 106F; another embodiment of the present invention, wherein said mutated phospholipase C enzyme comprises the amino acid substitution 121T; another embodiment of the present invention, wherein said mutated phospholipase C enzyme comprises the amino acid substitution

188P. Another embodiment of the present invention, wherein said mutated phospholipase C enzyme comprises the amino acid substitution 189K. Another embodiment of the present invention, wherein said mutated phospholipase C enzyme comprises the amino acid substitution 230I. Another embodiment of the present invention, wherein said mutated phospholipase C enzyme comprises the amino acid substitution
53D. Another embodiment of the present invention, wherein said mutated phospholipase C enzyme comprises the amino acid substitution 82E. Another embodiment of the present invention, wherein said mutated phospholipase C enzyme comprises the amino acid substitution 178E. Another embodiment of the present invention, wherein said mutated phospholipase C enzyme comprises the amino acid substitution 194K.

Another object of the present invention is to provide a mutated phospholipase C enzyme wherein its amino acid sequence comprises at least two amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention, the mutated phospholipase C enzyme of the invention wherein its amino acid sequence comprises at least three amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention is to provide a mutated phospholipase C enzyme according wherein its amino acid sequence comprises at least four amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention, is to provide a mutated phospholipase C enzyme wherein its amino acid sequence comprises at least five amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention is to provide a mutated phospholipase C enzyme wherein its amino acid sequence comprises at least six amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1. In a preferred embodiment, said mutated phospholipase C enzyme, wherein its substituted amino acids are in the positions 120F, 85N, 88T, 106F, 188P, 189K. In a more preferred embodiment said substitutions are L120F, Q85N, E88T, M106F, A188P, G189K. In another preferred embodiment of the present invention, wherein its substituted amino acids are in the positions 120F 85N, 88T, 106F, 121T and 230I.

Another object of the present invention is to provide a mutated phospholipase C enzyme wherein its amino acid sequence comprises at least seven amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention is to provide a mutated phospholipase C enzyme wherein its amino acid sequence comprises at least eight amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention is to provide a mutated phospholipase C enzyme according to claim 1, wherein its amino acid sequence comprises at least nine amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention is to provide a mutated phospholipase C enzyme wherein its amino acid sequence comprises at least ten amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention is to provide a mutated phospholipase C enzyme wherein its amino acid sequence comprises at least eleven amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

Another object of the present invention is to provide a mutated phospholipase C enzyme wherein its amino acid sequence comprises at least twelve amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1. In a preferred embodiment of the present invention, the mutated phospholipase C enzyme wherein said substituted amino acid are selecting form the group consisting of L120F, Q85N, E88T, M106F, G121T, A188P, G189K, V230I, A53D, Y82E, G178E and N194K.

Another object of the present invention is to provide a mutated phospholipase C enzyme wherein the said mutated phospholipase C enzyme comprises the amino acid sequence selected from de group comprising SEQ ID No. 2, SEQ ID No. 3, SEQ ID No. 4, SEQ ID No. 5, SEQ ID No. 6 and SEQ ID No. 11. In a preferred embodiment of the present invention, wherein said mutated phospholipase C enzyme the amino acid sequence SEQ ID No. 2 and wherein said the amino acid sequence SEQ ID No. 2 is encoding by the nucleotide acid sequence SEQ ID No. 10. In another preferred embodiment of the present invention, the mutated phospholipase C enzyme comprises the amino acid sequence SEQ ID No. 11; and said sequence is encoding by the nucleotide sequence SEQ ID No. 12.

The mutated phospholipase C enzyme, object of the present invention, wherein said enzyme is a thermostable at temperatures up to 85° C.

Another object of the present invention is to provide a procedure for oil degumming wherein said procedure comprises the following steps: i). adding a quantity of a mutated phospholipase C enzyme of claim 1 to a quantity of crude oil; and ii). incubate the reaction mixture at a temperature from 50° C. to 85° C. In a preferred embodiment of the present invention, wherein the quantity of mutated phospholipase C enzyme added in step i) comprises from 1 μg/g oil to 5 μg/g oil. Another preferred embodiment of the procedure is wherein said temperature in said step ii) comprises a temperature from 60° C. to 85° C. In a more preferred embodiment, said temperature is from 70° C. to 85° C. In another embodiment of the present procedure wherein a PI PLC is adding with the mutated phospholipase C enzyme. In a more preferred embodiment, said PI PLC comprises the amino acid sequence SEQ ID No. 9. In another embodiment of the procedure of the present invention, wherein said mutated phospholipase C enzyme of the step i) comprises an amino acid sequence wherein at least one amino acid is substituted in the position selected from the group consisting of L120F, Q85N, E88T, M106F, G121T, A188P, G189K, V230I, A53D, Y82E, G178E and N194K of the amino acid sequence of SEQ ID N. 1 or an amino acid sequence with at least 80% identical of SEQ ID No. 1. In a more preferred embodiment, said mutated phospholipase C enzyme is selected from de group comprising SEQ ID No. 2, SEQ ID No. 3, SEQ ID No. 4, SEQ ID No. 5, SEQ ID No. 6 and SEQ ID No. 11.

Another object of the present invention is to provide a use of a mutated phospholipase C enzyme, object of the present invention, for oil degumming at temperature between 50-85° C.; more preferred between 60-85° C., more preferred between 70-85° C.

### DESCRIPTION OF THE DRAWINGS

FIG. 1: Simplified diagram comparing (A) process of water degumming of industrial oil refining plants with (B) enzymatic degumming using enzymes at 55° C., and (C) the process of the present invention of enzymatic degumming, at temperature between 55° C. to 85° C., employing the thermostable enzymes of invention.

FIG. 2: PLC 596 and BC PLC (control) were pre incubated at room temperature, 60, 65, 70, 75 and 80° C. for 30 min before the PLC assay. The PLC activity was measured with O-(4-Nitrophenylphosphoryl) choline as substrate and Abs 405 nm was record at different time up to 15 min.

FIG. 3: PLC activity in soybean crude oil was measured as described in example 3. BC PLC (SEQ ID No. 1), PLC 596 (SEQ ID No. 2), PLC Novozymes (SEQ ID No. 7) and PLC Purifine (SEQ ID No. 8) were assayed in small scale degumming reactions for 120 min at different temperatures (50, 60, 70, 80 and 85° C.).

FIG. 4: PLC activity in soybean crude oil was measured as described in example 3. BC PLC (SEQ ID No. 1), PLC 596 (SEQ ID No. 2) and PLC Novozymes (SEQ ID No. 7) were assayed in small scale degumming reactions for 120 min at different temperatures (65, 70, 75, 80 and 85° C.).

FIG. 5: Crude soybean oil was treated with PLC 596 at different temperatures (50, 60, 70, 80, 85° C.) for 120 min. After, phospholipids were quantified by ³²P RMN. Values represent percentage remaining phospholipids relative to control sample (no enzyme added).

FIG. 6: Crude soybean oil was treated by BC PLC (SEQ ID No. 1) at different temperatures (50, 60, 70, 80, 85° C.) for 120 min. After, phospholipids were quantified by ³²P RMN. Values represent percentage remaining phospholipids relative to control sample (no enzyme added).

FIG. 7: Crude soybean oil was treated by BC PLC (SEQ ID No. 1), PLC 596 (SEQ ID No. 2) and PLC Novozymes (SEQ ID No. 7) at 80° C. for 120 min. After, phospholipids were quantified by ³²P RMN. Values represent percentage remaining phospholipids relative to control sample (no enzyme added).

FIG. 8: Crude soybean oil was treated by PLC 596 (SEQ ID No. 2) at 80° C. for 0, 30, 45 and 120 min. After, phospholipids were quantified by ³²P RMN. Values represent percentage remaining phospholipids relative to control sample (no enzyme added).

FIG. 9: PLC activity in soybean crude oil was measured. PLC 596 and PLC-596PP (SEQ ID No. 11) were assayed in small-scale degumming reactions for 120 min at different temperatures (65, 70, 75, 80 and 85° C.).

### DETAILED DESCRIPTION

According to the present invention, the term “enzyme” or “enzymes” should be understood as any polypeptide having phospholipase C activity. According to the present invention, the terms “mutated enzyme” and “Mutated phospholipase C enzyme” should be understood as any polypeptide having phospholipase C activity and having at least one substitution in particular positions defined according to the present invention.

In order to improve and optimize the degumming step, particularly improving and optimizing the enzymatic activity of phospholipase C enzymes (PLC), the inventors have designed, developed and produced new high-temperature resistant PLC enzymes, maintaining high enzymatic activity. More preferred, the PLC enzymes are phosphatidylcholine-specific phospholipase C (PC-PLC).

Through a consensus-based engineering method, starting from 14 natural sequences corresponding to the enzymes from mesophilic microorganisms, a large number of PLCs designed to withstand high temperatures were obtained that included at least one-point or substitution mutation in the sequences.

### State of Art Enzymes Vs Enzymes of the Invention in Degumming Step of Oil Extraction

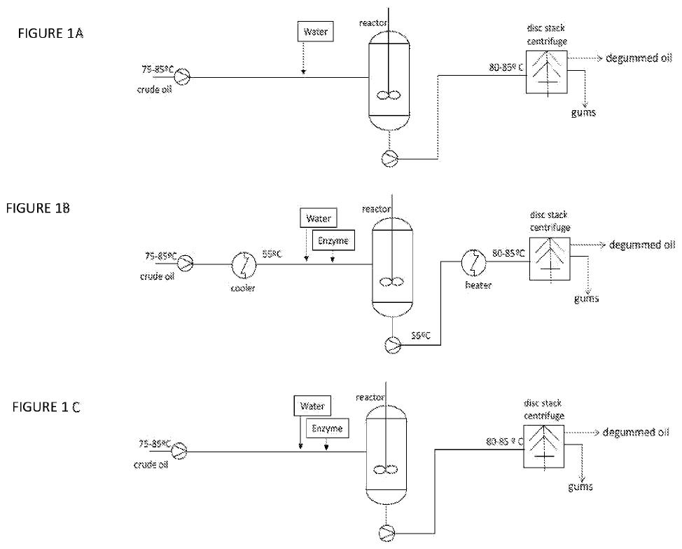

The FIG. 1A shows a simplified scheme of degumming step of the majority of industrial oil refining plants using water degumming. The oil is extracted from the flaked seeds using hexane, which is next distilled. The next step is water degumming. In the process, the extracted crude oil is mixed with 2-3% water and the water/oil emulsion pumped into the agitated tank at 75-80° C. The residence time is 35-40 minutes, the time required for the PLs to migrate to the water and form a heavy phase, known as gums, that is next separated by centrifugation to obtain degummed oil.

The FIG. 1B shows a simplified scheme of degumming step of industrial oil refining plants using enzymatic degumming. Today, the commercial PLCs works at 55° C. and require between 2 and 6 h to hydrolyze PLs (for example PLC Novozymes, SEQ ID No. 7), making necessary the expansion of the current plants, where supplementary heat exchangers before and after an additional large degumming tank, extra piping, pumps, tanks, mixers and control instruments need to be inserted between the hexane extraction units and the centrifuge used to separate the gums from the treated oil. A thermostable PLC, according to the present invention, capable of hydrolyzing the PLs would allow for the use of enzymatic degumming in the same facilities, by adding the enzymes to the water required to form the gums.

FIG. 1 C shows the procedure employing the thermostable PLCs enzymes of the invention, for high temperature oil degumming, where no extra equipment is required, and where the enzyme is dosed directly into the water used in the degumming process. After a 30 minutes' residence step, the same as the current aqueous degumming process, the emulsion is separated with a centrifuge and near 2% of extra oil is recovered, due to the miscible DAGs generated by the enzymes and the recovery of neutral oil that was trapped by the gums hydrolyzed by the thermostable PLC enzymes of the invention.

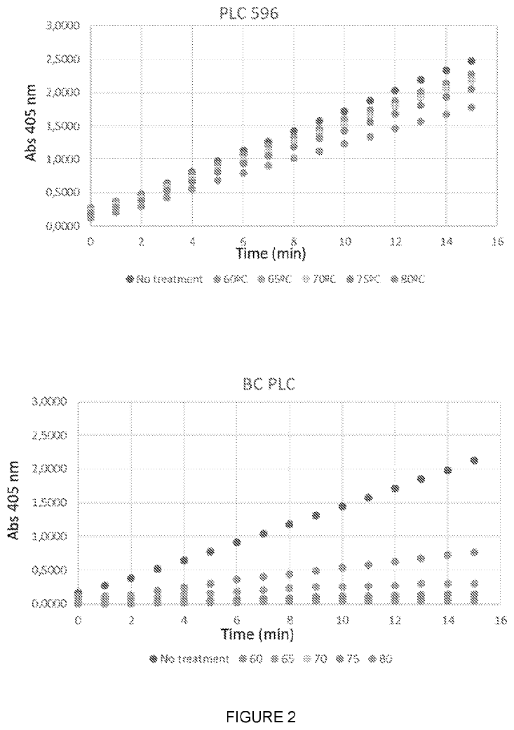

To evaluate protein thermal stability, purified BC-PLC (control, SEQ ID No. 1) and PLC-596 (SEQ ID No. 2) enzymes incubated at different temperatures (60-65-70-75-80° C.) for 30 minutes before PC-PLC activity was measured. FIG. 2 shows that PLC-596 (SEQ ID No. 2) retains more than 80% of its initial activity even after 30 min incubation at 75° C., and more than 70% after 30 min incubation at 80° C. In contrast BC PLC (SEQ ID No. 1) retained less than 10% of its initial activity after 30 min incubation at 70° C. or higher temperatures. The residual activity is shown in the Table 1. The PLC activity was measured with O-(4-Nitrophenylphosphoryl) choline as substrate as described in example 2.

TABLE 1

Residual enzymatic activity (%)

| | | PLC S96 | BC PLC |
|---|---|---|---|
| 30 min pre- | No treatment | 100 | 100 |
| treatment | 60° C. | 90 | 35 |
| | 65° C. | 91 | 14 |
| | 70° C. | 88 | 7 |
| | 75° C. | 83 | 8 |
| | 80° C. | 74 | 3 |

As shown in table 1, the residual enzymatic activity of PLC 596 (SEQ ID No. 2) was 70% at 80° C. and 30 minutes, while the enzymatic activity of BC PLC was 3% (SEQ ID No. 1). BC PLC already at 60° C. showed a significant reduction in the enzymatic activity.

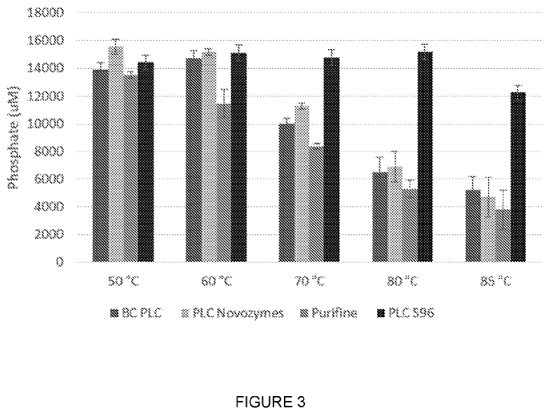

FIG. 3 shows that PLC 596 (SEQ ID No. 2) activity in soybean oil is high at 50, 60, 70 and 80° C. in contrast to two enzymes declared to be thermostable of the market which shows low values at temperatures higher than 60° C. The residual enzymatic activity shown with PLC 596 (SEQ ID No. 2) was also observed when tested the activity of PLC-596PP (SEQ ID No. 11), another embodiment of the invention. The difference between PLC 596 and PLC 596PP are 3 amino acids (N63, N131 and N134) that were mutated to improve the expression in Pichia Pastoris. PLC-596PP (SEQ ID No. 11) is PLC-596 (SEQ ID No. 2) containing N63D, N131S and N134D mutations. The nucleotide sequence SEQ ID No. 12 encodes the amino acid sequence of PLC-569PP.

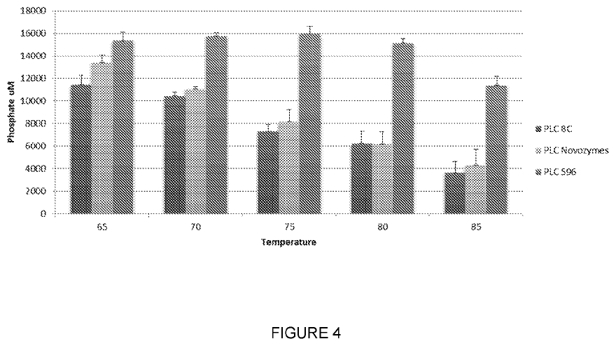

FIG. 4 shows that PLC-596 activity in soybean oil is maximum at 65, 70, 75 and 80° C. in contrast to the BC PLC (SEQ ID No. 1) and PLC Novozymes (SEQ ID No. 7) enzymes which show low values at temperatures higher than 60° C.

When the experiments described in example 5 were carried out (Small scale oil degumming experiments) the remaining phospholipids were shown quantified in relation to the amount of crude oil control sample (no enzymes added), Table 2: PLC 596, Table 3: BC PLC

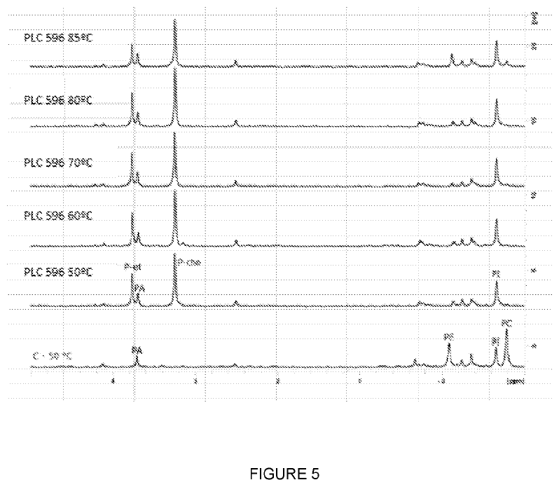

PLC-596 (SEQ ID No. 2) completely hydrolyzes PC (phosphatidylcholine) between 5° and 80° C. as no PC can be detected after treatment at these temperatures. Treatment at 85° C. results in 89.3% PC hydrolysis. PE (phosphatidylethanolamine) content is reduced by more than 90% with PLC-596 (SEQ ID No. 2) treatment at 50, 60, 70 and 80° C. (See Table 2 and FIG. 5).

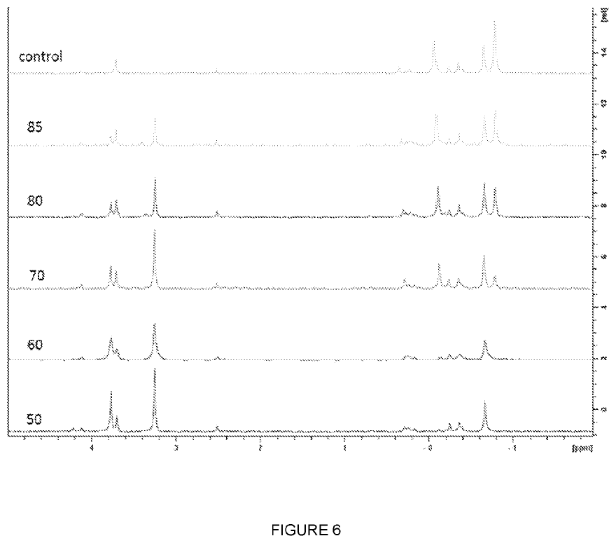

In contrast, BC PLC treatment is efficient only at 50 and 60° C. At 70° C. or higher, more than 50% PE is not hydrolyzed. 17.1% PC is not hydrolyzed at 70° C. and 40% or more PC is not hydrolyzed at 80° C. or higher. (See Table 3 and FIG. 6).

TABLE 2
Remaining phospholipid

| | No enzyme | PLC 596 | | | | |
|---|---|---|---|---|---|---|
| Temperature (° C.) | 50 | 50 | 60 | 70 | 80 | 85 |
| PC | 100 | 3.5 | ND | ND | ND | 10.7 |
| PI | 100 | 100 | 100 | 100 | 100 | 100 |
| PE | 100 | 6.9 | 5.2 | 4.7 | 7.1 | 32.7 |
| PA | 100 | 104.9 | 108.3 | 102.7 | 104.5 | 107.2 |

ND: not detected,
PC (phosphatidylcholine, PI (phosphatidylinositol), PE (phosphatidylethanolamine) and PA (phosphatidic acid)

TABLE 3
Remaining phospholipid

| | No enzyme | BC PLC | | | | |
|---|---|---|---|---|---|---|
| Temperature (° C.) | 50 | 50 | 60 | 70 | 80 | 85 |
| PC | 100 | ND | ND | 17.1 | 46.5 | 60.4 |
| PI | 100 | 100 | 100 | 100 | 100 | 100 |
| PE | 100 | 11.9 | 9.8 | 56.9 | 79.6 | 86.3 |
| PA | 100 | 100.5 | 93 | 91 | 103.5 | 104.9 |

ND: not detected,
PC (phosphatidylcholine, PI (phosphatidylinositol), PE (phosphatidylethanolamine) and PA (phosphatidic acid)

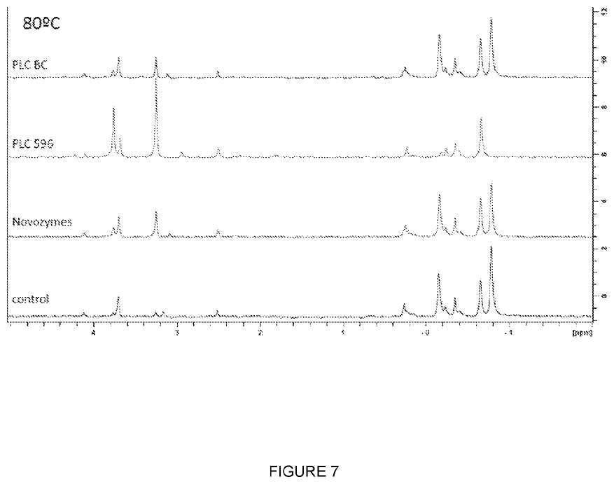

PLC 596 (SEQ ID No. 2) completely hydrolyzes PC and more than 90% PE after 120 min treatment at 80° C. In contrast, BC PLC and PLC Novozymes (SEQ ID No. 7) treatment are not efficient at this temperature, wherein more than 70% of PL were not hydrolyzed. The Table 4 and FIG. 7 show the remaining phospholipids after treatment at 80° C.

TABLE 4
Remaining phospholipid

| No | BC | PLC | PLC |
|---|---|---|---|
| enzyme | PLC | 596 | Novozymes |

| | |
|---|---|
| Temperature (° C.) | 80 |

| | | | | |
|---|---|---|---|---|
| PC | 100 | 80.8 | ND | 71.5 |
| PI | 100 | 100 | 100 | 100 |
| PE | 100 | 94.7 | 7.2 | 92.5 |
| PA | 100 | 97.8 | 93.4 | 98.2 |

ND: not detected,
PC (phosphatidylcholine, PI (phosphatidylinositol), PE (phosphatidylethanolamine) and PA (phosphatidic acid)

PLC 596 completely hydrolyzes PC and more than 90% PE after treatment at 80° C. even at shorter times (30 or 45 min) with a lower enzyme dose (3 μg enzyme/g oil).

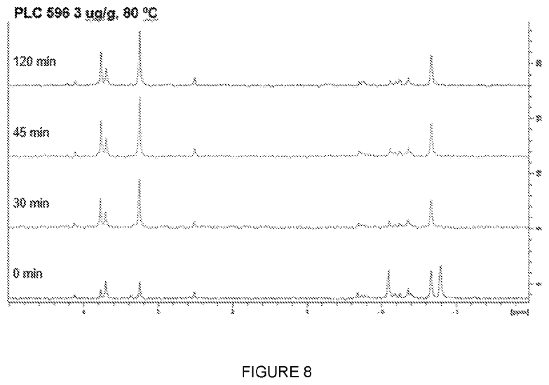

Remaining phospholipids were quantified relative to the amount in crude oil control sample (no enzymes added) (See Table 5 and FIG. 8).

TABLE 5
Remaining phospholipid

| | PLC 596 |
|---|---|
| Temperature (° C.) | 80 |

| | | | | |
|---|---|---|---|---|
| Time (min) | 0 | 30 | 45 | 120 |
| PC | 100 | ND | ND | ND |
| PI | 100 | 100 | 100 | 100 |
| PE | 100 | 7.2 | 5.1 | 3.3 |
| PA | 100 | 98.7 | 97.5 | 102 |

ND: not detected,
PC (phosphatidylcholine, PI (phosphatidylinositol), PE (phosphatidylethanolamine) and PA (phosphatidic acid)

PLC 596 (SEQ ID No. 2) completely hydrolyzes PC and more than 90% PE after treatment at 80° C. during 30 minutes with different enzyme doses (from 2 to 5 μg enzyme/g oil). Remaining phospholipids were quantified relative to the amount in crude oil control sample (no enzymes added). (See, Table 6).

TABLE 6
Remaining phospholipid

| | PLC 596 |
|---|---|
| Temperature (° C.) | 80 |
| Time (min) | 30 |

| | | | | |
|---|---|---|---|---|
| ug Enzyme/g of oil | 5 | 3 | 2 | 1 |
| PC | ND | ND | 2.8 | 7.3 |
| PI | 100 | 100 | 100 | 100 |
| PE | ND | 5.3 | 7.1 | 12.4 |
| PA | 100 | 99.7 | 98.5 | 101.4 |

ND: not detected,
PC (phosphatidylcholine, PI (phosphatidylinositol), PE (phosphatidylethanolamine) and PA (phosphatidic acid)

The PLC 596 (SEQ ID No. 2) of the invention can be combined with other phospholipases, like PI-PLC 455 (SEQ ID No. 9), and the combination hydrolyzes more than the 95% of the PC, PI and PE present in the sample oil.

Remaining phospholipids were quantified relative to the amount in crude oil control sample (no enzymes added) (See Table 7).

TABLE 7
Remaining phospholipid

| | Enzymes | | |
|---|---|---|---|
| | PLC 596 3 ug/g of oil | | |
| | PI PLC 455 1 ug/g of oil | | |
| Temperature (° C.) | 60 | | |
| Time(min) | 0 | 30 | 120 |
| PC | 100 | 3.4 | ND |
| PI | 100 | 2.7 | ND |
| PE | 100 | 5.3 | 3.7 |
| PA | 100 | 99.7 | 98.5 |

ND: not detected,
PC (phosphatidylcholine, PI (phosphatidylinositol), PE (phosphatidylethanolamine) and PA (phosphatidic acid)

The present invention is further described by the following examples that should not be considered as limiting the scope of the invention.

### EXAMPLES

### Example 1—PLC Design, Expression and Purification

Synthetic PLC enzymes were designed in silico using consensus-based engineering starting from 14 natural sequences corresponding to the enzymes from mesophilic microorganisms (Table 8, incorporated here as references):

TABLE 8

| NCBI Reference | UniProtKB/ | | |
|---|---|---|---|
| Sequence | Swiss-Prot: | Microorganism | Protein name |
| WP_000731014.1 | P09598 PHLC_BACCE | Bacillus cereus | Seq_1 |
| | | | Bacillus cereus |
| WP_040119128.1 | A0A076W6F1_BACMY | Bacillus | A0A076W6F1\|A0A076W6F1_BACMY |
| | | pseudomycoides | Phospholipase C domain |
| | | | protein OS = Bacillus mycoides |
| WP_007203237.1 | I8AFV4_9BACI | Fictibacillus | I8AFV4\|I8AFV4_9BACI |
| | | macauensis | Phospholipase C OS = |
| | | | Bacillus macauensis |
| WP_042985025.1 | A0A090Y8D7_BACMY | Bacillus clarus | A0A090Y8D7\|A0A090Y8D7_BACMY |
| | | | Phospholipase C domain |
| | | | protein OS = Bacillus mycoides |
| WP_000823154.1 | J8RQ87_BACCE | Bacillus cereus | J8RQ87\|J8RQ87_BACCE |
| | | | Phospholipase C OS = |
| | | | Bacillus cereus BAG2X1-1 |
| WP_034641782.1 | A0A073JVA0_9BACI | Bacillus | A0A073JVA0\|A0A073JVA0_{—} |
| | | manliponensis | 9BACI Phospholipase C |
| | | | OS = Bacillus manliponensis |
| GenBank: | A0A0K1PJ70_9DELT | Labilithrix luteola | A0A0K1PJ70\|A0A0K1PJ70_9DELT |
| AKU93446.1 | | | Broad-substrate range |
| | | | phospholipase C OS = |
| | | | Labilithrix luteola |
| GenBank: | C3GBR0_BACTU | Bacillus | C3GBR0\|C3GBR0_BACTU |
| EEM68651.1 | | thuringiensis | Phospholipase C OS = |
| | | serovar | Bacillus thuringiensis serovar |
| | | andalousiensis | andalousiensis |
| | | BGSC 4AW1 | BGSC 4AW1 |
| GenBank: | A0A0A0WUN9_9BACI | Bacillus mycoides | A0A0A0WUN9\|A0A0A0WUN9_9BACI |
| EEL03127.1 | | | Phospholipase C |
| | | | OS = Bacillus |
| | | | weihenstephanensis |
| WP_003718282.1 | Q6R6C7_LISIV | Listeria ivanovii | Q6R6C7\|Q6R6C7_LISIV |
| | | | Phospholipase C OS = Listeria |
| | | | ivanovii subsp. londoniensis |
| GenBank: | Q84DK1_LISSE | Listeria seeligeri | Q84DK1\|Q84DK1_LISSE |
| AAO19486.1 | | | Phospholipase (Fragment) |
| | | | OS = Listeria seeligeri |
| WP_070034811.1 | B9UY68_LISMN | Listeria | B9UY68\|B9UY68_LISMN |
| | | monocytogenes | Phospholipase C OS = |
| | | | Listeria monocytogenes |
| WP_000725200 | J8LVK6_BACCE | Bacillus cereus | J8LVK6\|J8LVK6_BACCE |
| | | BAG1O-2 | Uncharacterized protein |
| | | | OS = Bacillus cereus BAG1O-2 |
| WP_003198006 | A0A0B6A4A9_BACCE | Bacillus cereus | A0A0B6A4A9\|A0A0B6A4A9_BACCE |
| | | | Phospholipase C |
| | | | OS = Bacillus cereus |

The selected sequences were chosen based on two criteria. First, the presence of a W residue at the N-terminal of the mature protein, and second, the presence of residues that can coordinate a Zn atom in the active site (for example H14, D55, H69, H118, D122, H128, H142, E146). The sequences were aligned and a consensus polypeptide designed by choosing for each position the amino acid occurring more frequently in the group of parental sequences.

### Protein Expression and Purification:

The synthetic DNA sequences encoding for the PLCs (PLC-596 (SEQ ID No. 2), PLC-596PP (SEQ ID No. 11), BC-PLC (SEQ ID No. 1), PLC Novozymes (SEQ ID No. 7) and PI-PLC 455 (SEQ ID No. 9) were inserted (NdeI-EcoRI) into the pTGR vector and expressed as secreted protein in batch cultures of C. glutamicum as previously described (Ravasi P., (2012) Microbial cell factories 11, 147; Ravasi, P., (2015) Journal of biotechnology 216, 142-148). Then, 500 mg of purified protein per liter of broth were obtained after ammonium sulfate precipitation and HIC chromatography purification.

The synthetic DNA sequences encoding for Purifine PLC (SEQ ID No. 8) was cloned into XhoI-XbaI restriction sites of the pPICZαA vector (Invitrogen). The resulting plasmid was linearized with SacI and transformed by electroporation into Pichia pastoris cells. Transformants were selected on YPD supplemented with zeocin 100 μg/ml. 100 colonies were streaked on PLC activity plates (YP 5% egg yolk, 0.5% methanol, 1 mM ZnSO₄, 1.5% agar) and colonies displaying the largest halos were selected for further analysis.

Fermentation of Pichia pastoris strain expressing the corresponding enzyme was performed according to the Invitrogen protocol for mut+ strains. The culture medium used is 1 liter of Fermentation Basal Salts Medium (BSM) pH 5 and cultures were grown at 30° C. in an Infors LabFors bioreactor with 2 liters of working volume.

The process starts with a 16 h batch phase followed by 3 hs of fed batch where the 10 feeding rate is 18.12 ml/h·L of glycerol 50% W/V+1.2% PTM1. Next, a methanol feeding phase of 40 h induces the expression of the enzymes. The feeding rates (methanol 100%+1.2% PTM1) in the induction phase is 3.6 ml/h·L for the first 2 h, 7.6 ml/h·L for 2 additional hours and 10.9 ml/h·L until the end of the process. The typical process yield is 5 g/L of secreted protein, a final OD₆₀₀ of 600, and an overall PLC volumetric 15 productivity of 3100 Units/L·h. Pichia supernatant was microfiltered and concentrated using a 10 KDa ultrafiltration cartridge.

### Example 2: PC-PLC Activity in Aqueous Buffer, Thermal Inactivation of PLC Enzymes

To evaluate protein thermal stability, purified BC-PLC (control) and PLC-596 enzymes were incubated at different temperatures (60-65-70-75-80° C.) for 30 minutes before PC-PLC activity was measured. After this incubation, proteins were cooled to room temperature (25° C.) and the PC-PLC activity was assayed.

Briefly, 10 μl of sample containing purified BC-PLC (control) and PLC-596 enzymes were incubated with 10 mM O-(4-Nitrophenylphosphoryl) choline as a substrate in buffer 250 mM HEPES pH7, 0.1 mM ZnCl₂ in a final volume of 100 μl at 25° C. for 15 min. Absorbance at 405 nm determined as a function of time. See FIG. 2 and table 1.

### Example 3: Oil Degumming at 50-85° C.

Oil degumming experiments were performed using BC PLC (control) (SEQ ID No 1), PLC 596 (SEQ ID No 2) and two commercially available enzymes: Purifine (SEQ ID No. 8) (DSM) and PCPLC (SEQ ID No: 7) (Novozymes).

Briefly, 3 g of crude soybean oil containing about 1000 ppm phosphate were homogenized for 1 min using Ultra-Turrax T8 Homogenizer (IKA) with 15 ug of each enzyme (BC PLC (control, SEQ ID No. 1), PLC 596 (SEQ ID No. 2), Purifine (DSM, SEQ ID No: 8) and PCPLC (Novozymes, SEQ ID No. 7) in 90 μl of water, (5 ug enzyme/g of oil). Next, the tubes containing the reaction mixture were incubated for 120 min at the indicated temperature (50-60-70-80-85° C.) with constant agitation using a magnetic tumble stirrer such as the VP 710 magnetic tumble stirrer (VP-Scientific).

Quantification of inorganic phosphate generated from polar heads groups of hydrolyzed phospholipids was used as a direct measure of PLC activity. After 120 min incubation, the oil was homogenized and 200 μl of the homogenized oil were mixed with 200 ul of 2 M Tris-HCl pH 8 to stop the reaction. Then, 800 μl of water were added to the mixture and incubated for 1 h at 37° C. with constant agitation, and then centrifuged for 5 min at 14000 g. Finally, 45 μl of the aqueous phase was recovered and treated with 0.3 U of calf intestinal phosphatase (Promega, WI, USA) for 1 h at 37° C.

The concentration of inorganic phosphate was determined according to the method of Sumner (Sumner, J. B., Science 1944 196:413). Briefly, a 500 μl sample, containing 0.025 to 0.25 μmol of inorganic phosphate in 5% TCA was mixed with 500 μl of color reagent (4% FeSO₄, 1% (NH₄)₆MoO₂₄·H₂O, 3.2% H₂SO₄). Spectrophotometric readings were made at 700 nm, and the micromoles of inorganic phosphate in the sample calculated using a standard curve. Results in FIG. 3 show that PLC 596 (SEQ ID No. 2) activity in soybean oil is maximum at 50, 60, 70 and 80° C. in contrast to the other PC PLC enzymes which show low values at temperatures higher than 60° C.

### Example 4: Oil Degumming at 65-85° C.

Small scale oil degumming experiments were performed as described in Example 3, using BC PLC (SEQ ID No. 1), PLC 596 (SEQ ID No. 2) and PCPLC (Novozymes, SEQ ID No. 7) enzymes (5 ug enzyme/g oil) and incubating the reaction mixture at 65-70-75-80-85° C. for 120 min.

Quantification of inorganic phosphate generated from polar heads groups of hydrolyzed phospholipids was used as a direct measure of PLC activity and was performed as indicated in Example 3. Results in FIG. 4 show that PLC 596 (SEQ ID No. 2) activity in soybean oil is maximum at 65, 70, 75 and 80° C. in contrast to the BC PLC (SEQ ID No. 1) and PLC Novozymes (SEQ ID No. 7) enzymes which show low values at temperatures higher than 60° C.

### Example 5: Small Scale Oil Degumming Experiments

Small scale oil degumming experiments were performed as indicated in Example 3, using BC PLC and PLC 596 (5 ug enzyme/g oil), incubating at 50-60-70-80-85° C. for 120 min.

After 120 min incubation at the indicated temperature, remaining phospholipids were characterized by NMR: Oil samples were extracted with 900 μl of NMR solution (100 mM Tris-HCl pH 10.5, 50 mM EDTA, 2.5% sodium deoxycholate) during 1 h at 37° C. with constant agitation step. The resulting aqueous phase were extracted with 600 μl hexane and then analyzed by NMR analysis.

NMR spectra of the crude oil and treated crude oil were acquired using a Bruker DRX 600 and samples of pure phosphatidylcholine (PC), phosphatidylethanolamine (PE), phosphatidic acid (PA) and phosphatidylinositol (PI) control run as standards. The results are shown in Table 2 and FIG. 5, and Table 3 and FIG. 6.

### Example 6

Small scale oil degumming experiments were performed as indicated in example 3, using BC PLC (SEQ ID No. 1), PLC 596 (SEQ ID No. 2) and PLC Novozymes (SEQ ID No. 7) (5 ug enzyme/g oil) incubating at 80° C. for 120 min.

After 120 min incubation at 80° C., oil samples were extracted with 900 μl of NMR solution (100 mM Tris-HCl pH 10.5, 50 mM EDTA, 2.5% sodium deoxycholate) during 1 h at 37° C. with constant agitation step. The resulting aqueous phase were extracted with 600 μl hexane and then analyzed by NMR analysis. Results are shown in Table 4.

NMR spectra of the crude oil and treated crude oil were acquired using a Bruker DRX 600 and samples of pure phosphatidylcholine (PC), phosphatidylethanolamine (PE), phosphatidic acid (PA) and phosphatidylinositol (PI) control run as standards. Results are shown in FIG. 7

### Example 7

Small scale oil degumming experiments were performed as indicated in example 3, using PLC 596 (SEQ ID No. 2) using a lower dose of enzyme, 3 ug enzyme/g oil, incubating at 80° C. for 0, 30, 45 and 120 min. Results shows in table 5.

After the indicated time incubation at 80° C., oil samples were extracted with 900 μl of NMR solution (100 mM Tris-HCl pH 10.5, 50 mM EDTA, 2.5% sodium deoxycholate) during 1 h at 37° C. with constant agitation step. The resulting aqueous phase were extracted with 600 μl hexane and then analyzed by NMR analysis (see FIG. 8).

NMR spectra of the crude oil and treated crude oil were acquired using a Bruker DRX 600 and samples of pure phosphatidylcholine (PC), phosphatidylethanolamine (PE), phosphatidic acid (PA) and phosphatidylinositol (PI) control run as standards.

### Example 8

Small scale oil degumming experiments were performed using PLC 596 (5, 3, 2, 1 μg enzyme/g oil) incubating at 80° C. for 30 min. (See Table 6)

After the indicated time incubation at 80° C., oil samples were extracted with 900 μl of NMR solution (100 mM Tris-HCl pH 10.5, 50 mM EDTA, 2.5% sodium deoxycholate) during 1 h at 37° C. with constant agitation step. The resulting aqueous phase were extracted with 600 μl hexane and then analyzed by NMR analysis.

NMR spectra of the crude oil and treated crude oil were acquired using a Bruker DRX 600 and samples of pure phosphatidylcholine (PC), phosphatidylethanolamine (PE), phosphatidic acid (PA) and phosphatidylinositol (PI) control run as standards.

### Example 9: PLC 596 in Combination with PI PLC 455

Small scale oil degumming experiments were performed using PLC 596 (SEQ ID No. 2) (3 ug enzyme/g oil) alone or in combination with PI PLC 455 (SEQ ID No. 9) (0.5-1 ug enzyme/g oil), incubating at 60° C. for 0, 30, and 120 min. Results are show in table 7.

After the indicated time incubation at 60° C., oil samples were extracted with 900 μl of NMR solution (100 mM Tris-HCl pH 10.5, 50 mM EDTA, 2.5% sodium deoxycholate) during 1 h at 37° C. with constant agitation step. The resulting aqueous phase were extracted with 600 μl hexane and then analyzed by NMR analysis.

NMR spectra of the crude oil and treated crude oil were acquired using a Bruker DRX 600 and samples of pure phosphatidylcholine (PC), phosphatidylethanolamine (PE), phosphatidic acid (PA) and phosphatidylinositol (PI) control run as standards.

### Example 10: Oil Degumming Experiments Using PLC-596PP (SEQ ID No. 11)

An oil degumming experiments were performing using enzyme sequence SEQ ID No. 11. Said enzyme were expressed according to example 1 and 2 and the oil degumming experiment were performed according to example 3.

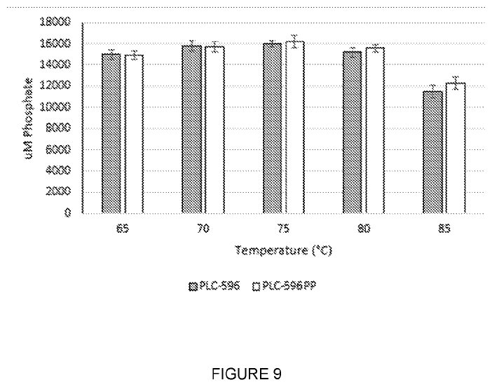

Results in FIG. 9 shows that PLC-596PP activity in soybean oil is maximum at 70-80° C., showing similar activity to PLC-596.

1.-47. (canceled)

48. A mutated phospholipase C enzyme comprising an amino acid sequence wherein at least one amino acid is substituted in a position selected from the group consisting of 120, 85, 88, 106, 121, 188, 189, 230, 53, 82, 178 and 194 of: the amino acid sequence of SEQ ID No. 1 or an amino acid sequence with at least 80%, 85%, 90%, 95%, 97% or 98% identical of SEQ ID No. 1.

49. The mutated phospholipase C enzyme according to claim 48, wherein said amino acid substitution is selected from the group consisting of 120F, 85N, 88T, 106F, 121 T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

50. The mutated phospholipase C enzyme according to claim 49, wherein said amino acid substitution is selected from the group consisting of L120F, Q85N, E88T, M106F, G121T, A188P, G189K, V230I, A53D, Y82E, G178E and N194K of: the amino acid sequence of SEQ ID No. 1 or an amino acid sequence with at least 80%, 85%, 90%, 95%, 97% or 98% identical of SEQ ID No. 1.

51. The mutated phospholipase C enzyme according to claim 48, wherein the amino acid sequence comprises at least two amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

52. The mutated phospholipase C enzyme according to claim 48, wherein the amino acid sequence comprises at least six amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

53. The mutated phospholipase C enzyme, according to claim 52, wherein the substituted amino acids are in the positions 120F, 85N, 88T, 106F, 188P, 189K.

54. The mutated phospholipase C enzyme according to claim 53, wherein said substituted amino acid are L120F, Q85N, E88T, M106F, A188P, G189K.

55. A mutated phospholipase C enzyme, according to claim 52, wherein the substituted amino acids are in the positions 120F 85N, 88T, 106F, 121T and 230I.

56. The mutated phospholipase C enzyme according to claim 48, wherein the amino acid sequence comprises at least eight amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

57. The mutated phospholipase C enzyme according to claim 48, wherein the amino acid sequence comprises at least twelve amino acid substitutions in the positions selected from the group consisting of 120F, 85N, 88T, 106F, 121T, 188P, 189K, 230I, 53D, 82E, 178E and 194K of: the amino acid sequence of SEQ ID No. 1 or an amino acid sequence with at least 80%, 85%, 90%, 95%, 97% or 98% identical of SEQ ID No. 1.

58. The mutated phospholipase C enzyme according to claim 57, wherein said substituted amino acid are selecting form the group consisting of L120F, Q85N, E88T, M106F, G121T, A188P, G189K, V230I, A53D, Y82E, G178E and N194K.

59. The mutated phospholipase C enzyme according to claim 48, wherein the said mutated phospholipase C enzyme comprises the amino acid sequence selected from de group comprising SEQ ID No. 2, SEQ ID No. 3, SEQ ID No. 4, SEQ ID No. 5, SEQ ID No. 6 and SEQ ID No. 11.

60. The mutated phospholipase C enzyme according to claim 59, wherein the said mutated phospholipase C enzyme comprises the amino acid sequence SEQ ID No. 2.

61. A nucleotide acid sequence encoding the polypeptide according to claim 60, wherein said nucleotide acid sequence is SEQ ID No. 10.

62. The mutated phospholipase C enzyme according to claim 59, wherein the said mutated phospholipase C enzyme comprises the amino acid sequence SEQ ID No. 11.

63. A nucleotide acid sequence encoding the polypeptide according to claim 62, wherein said nucleotide acid sequence is SEQ ID No. 12.

64. A procedure for oil degumming wherein said procedure comprises the following steps:
i) adding a quantity of a mutated phospholipase C enzyme of claim 48 from 1 pg/g oil to 5 pg/g oil; and
ii) incubating the reaction mixture at a temperature from 50° C. to 85° C.; optionally adding a PI PLC with the mutated phospholipase C enzyme.

65. The procedure according to claim 64, wherein the mutated phospholipase C enzyme, comprises an amino acid sequence wherein at least one amino acid is substituted in the position selected from the group consisting of L120F, Q85N, E88T, M106F, G121T, A188P, G189K, V230I, A53D, Y82E, G178E and N194K of the amino acid sequence of SEQ ID N. 1 or an amino acid sequence with at least 80% identical of SEQ ID No. 1.

66. The procedure according to claim 64, wherein the mutated phospholipase C enzyme is selected from de group comprising SEQ ID No. 2, SEQ ID No. 3, SEQ ID No. 4, SEQ ID No. 5, SEQ ID No. 6 and SEQ ID No. 11.
